(19) United States
(12) Patent Application Publication
(10) Pub. No.: US 2024/0071618 A1
(43) Pub. Date: Feb. 29, 2024

(54) INCREASED ACCURACY OF RELAYED MEDICAL INFORMATION

(71) Applicant: Charu Software Solutions, Jacksonville, FL (US)

(72) Inventors: Charu G. Raheja, Winston-Salem, NC (US); Ravi K. Raheja, Nashville, TN (US)

(21) Appl. No.: 18/455,496

(22) Filed: Aug. 24, 2023

Related U.S. Application Data
(60) Provisional application No. 63/373,419, filed on Aug. 24, 2022.

(51) Int. Cl. G16H 50/20; G16H 10/20

(52) U.S. Cl. CPC G16H 50/20; G16H 10/20

(57) ABSTRACT

A system and method are disclosed to enhance the efficiency and accuracy of medical information relayed to medical providers for real-time clinical decision support, using algorithmic techniques to automatically ask additional related questions to improve and enhance provider and patient communications. The present subject matter generally relates to improving medical safety, efficiency, and time management, reducing medical malpractice and system errors, and more particularly, to a system and method for intelligent and enhanced questions to be answered to submit to the provider for clinical actions. Among the many benefits provided by the subject matters disclosed herein are improved medical diagnostic of patient symptoms, service efficiency, and improved patient experience and satisfaction, all of which improve healthcare operations and patient outcomes in settings where a message about a patient's symptom is being related to the practitioner by the patient or a non-clinical agent.

### PRIORITY APPLICATION

This application claims priority to U.S. Provisional Patent Application Ser. No. 63/373,419, filed on Aug. 24, 2022, the disclosure of which is incorporated by reference herein in its entirety.

### TECHNICAL FIELD

Embodiments described herein generally relate to conveying medical information.

### BACKGROUND

Relaying medical messages is marked by inefficiencies and archaic practices that have changed little over the last several decades, despite all the medical advances over that same period. When a patient experiences a symptom, the medical practitioner (e.g., physician) is typically not immediately available by phone, so a message is left by the patient with a receptionist or answering service, and the message is subsequently conveyed to the medical practitioner. However, the message may not include all of the needed information, and may not indicate the severity of the symptom.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not limitation, in the figures of the accompanying drawings in which:

FIG. 1 is a flowchart illustrating a first medical message relay 100, according to an embodiment.

FIG. 2 is a flowchart illustrating a second medical message relay 200, according to an embodiment.

FIG. 3 is a flowchart diagram of a message relay method 300, according to an embodiment.

FIG. 4 is a block diagram of a computing device 400, according to an embodiment.

### DETAILED DESCRIPTION

Technical solutions are described herein to address technical problems facing relaying medical messages. These technical problems may include medical messages that do not contain enough information for a medical service provider to provide medical care, do not contain enough information for a medical service provider to determine a medical event severity, or do not address related symptoms that may be time-sensitive or life-threatening. As described herein, technical solutions to these technical problems may include improving efficiency and accuracy of medical information relayed to medical providers for real-time clinical decision support by using algorithmic techniques to identify additional needed medical information, prompt a patient to provide the additional information, and analyze the provided responses and stored medical information to correctly identify the medical event, to identify the medical event severity, and to convey the medical event information and severity to a medical practitioner. These algorithmic techniques may include deterministic techniques (e.g., following a flowchart), probabilistic techniques (e.g., statistical predictions), heuristic techniques (e.g., rapid approximations), and other algorithmic techniques.

The present subject matter generally relates to improving medical safety, efficiency, and time management, reducing medical malpractice and system errors, and more particularly, to a system and method for intelligent and enhanced questions to be answered to submit to the provider for clinical actions. Among the many benefits provided by the subject matters disclosed herein are improved medical diagnostic of patient symptoms, improved service efficiency, and improved patient experience and satisfaction, all of which improve healthcare operations and patient outcomes in settings where a message about a patient's symptom is being related to the practitioner by the patient or a non-clinical agent.

The use of algorithmic techniques may provide various advantages over other solutions, such as solutions using artificial intelligence (AI). By using algorithmic techniques, these technical solutions may provide more visibility and insight into the decision-making process for a medical practitioner. These algorithmic techniques may avoid various pitfalls associated with AI, such as avoiding inheriting biases from biased historical data and avoiding the lack of explainability or repeatability of certain AI models.

By improving efficiency and accuracy of medical information relayed to medical providers, these technical solutions improve the operation of computing devices and medical computing systems (e.g., electronic medical record (EMR systems). By using advanced computer processing and communications to receive a medical message, analyze the medical message, and automatically request additional information and determine a severity or urgency, these solutions improve computer processing by enabling more intelligent analysis of medical messages. These technical solutions apply algorithmic techniques (e.g., probabilistic techniques, heuristic techniques, and deterministic techniques) to analyze medical messages and generate relevant follow-up questions, this allows the computing devices and medical computing systems to make more informed decisions. These technical solutions further use rules engines to determine urgency levels based on medical messages and additional patient information, which improves the ability of computing devices and medical computing systems to generate accurate assessments and reduce duplicated or redundant computer processing that may otherwise be used to collect additional information. These technical solutions generate personalized questions tailored to the patient, medical provider preferences, and medical coding systems, this improves the relevance of the medical information and the adaptability of the computing devices and medical computing systems. These technical solutions may be implemented to collect the patient information and generate additional questions using a non-medical practitioner (e.g., telehealth operator) or patient messaging service, and these additional features expand the capabilities of the computing devices and medical computing systems. Taken together, the use of advanced computer processing, analysis, and communications in these technical solutions provide improved accuracy, efficiency, and capabilities of computing systems that relay medical information.

In the following description, for purposes of explanation, numerous specific details are set forth to provide a thorough understanding of some example embodiments. It will be evident, however, to one skilled in the art that the present disclosure may be practiced without these specific details.

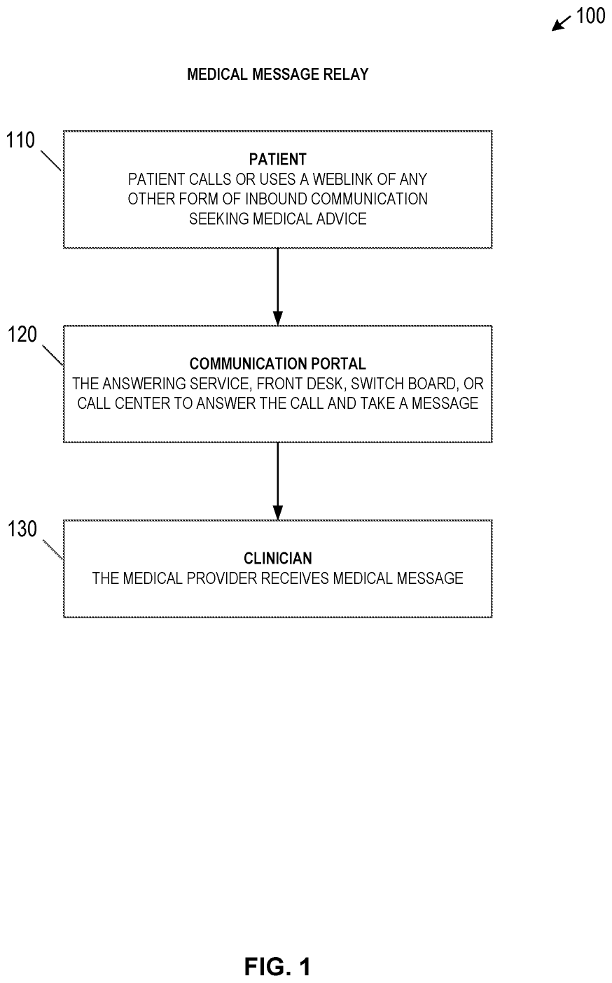

FIG. 1 is a flowchart illustrating a first medical message relay 100, according to an embodiment. The first medical message relay 100 depicts a first process for medical messaging. Initially, a patient 110 experiences a symptom and leaves a message for the physician, and medical communication portal 120 (e.g., medical answering service, contact center) taking the message may record text indicating the reason for the call. This information may be relayed to a clinician 130, who may contact the patient 110 to request more information or to take additional steps as needed to address the recorded symptoms.

To provide improved relaying of medical information, an improved medical message relay system may receive the message, and the system may use advanced computer processing and communications to request additional information to identify the medical event or determine a severity level. For example, if a patient calls asking for a prescription refill, the patient may be prompted to answer if they are having additional symptoms. The system may prompt the patient with one or more questions to determine whether additional information is needed for a medical practitioner to provide medical care for the additional symptoms. The system may also prompt the patient with one or more questions to determine the severity (e.g., seriousness) of the call, and may further prompt the patient with one or more questions to determine the urgency in replying to the patient or in addressing the patient's symptoms. In an example, this may be used to reduce or prevent a delay in responding to a patient who called for a prescription refill but is also experiencing vertigo, heart rhythm irregularity, or another a serious symptom.

The improved medical message relay system may provide improved telephone triage for medical messages, such as by generating questions during the call to identify the medical event automatically and correctly, to determine severity, to determine response urgency, and to determine other related characteristics related to the medical event. In an example, the improved medical message relay system may use algorithmic techniques to analyze the text of the message and prompt for additional quick clarifying questions. The improved medical message relay system may prompt the patient to answer these additional questions, such as by prompting a patient with text when using an automated system (e.g., online messaging, text message, instant message), or may prompt a human operator to ask the additional questions to the patient. This improved medical message relay system may use algorithmic techniques to identify needed information and associated questions, such as diverse types of deterministic or probabilistic flowcharts. Once additional needed information has been identified, this improved medical message relay system may use additional technical means to generate questions, such as diverse types of generative artificial intelligence, machine learning, expert systems, and internet-based communications to generate questions to prompt for the needed information. In an example, the questions may be tailored based on the preferences of the human operator, for the preferences of the patient, for the medical coding system, or based on other considerations. This identification of needed information and generation of associated questions may be used to increase the efficiency of medical provider and patient communications. Significantly, this improved medical message relay system may enable a human operator to get enough information to determine the medical event, the medical event severity, or the medical event urgency without requiring the human operator to be a trained medical professional. This also allows an automated system to determine medical event severity or urgency.

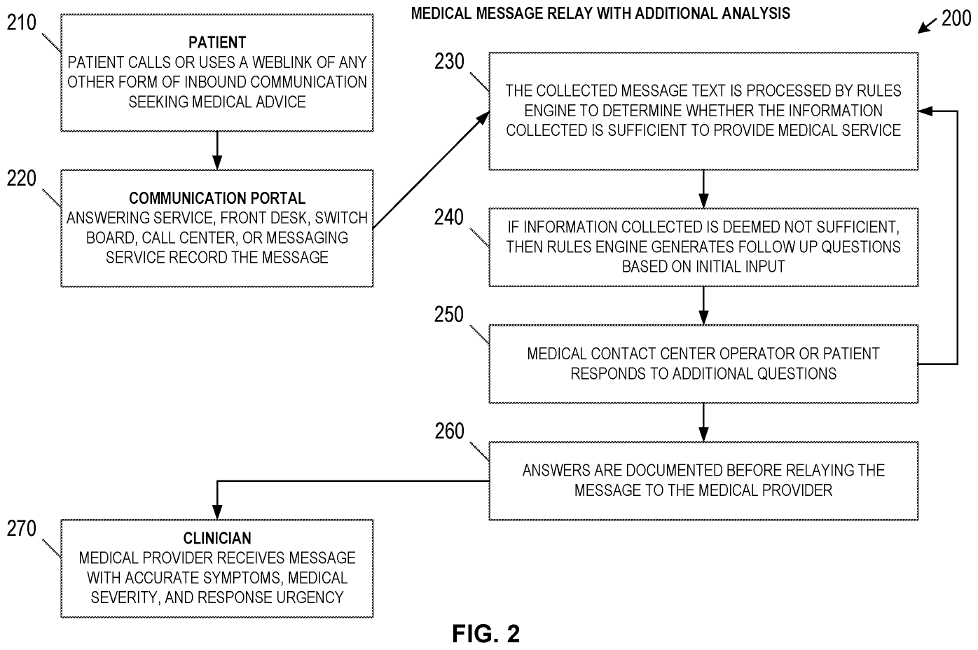

FIG. 2 is a flowchart illustrating a second medical message relay 200, according to an embodiment. Initially, a patient 210 experiences a symptom and communicates a message for the physician, and the communication portal 220 interacting with the patient may record text indicating the information provided by the patient. The communication portal 220 may also record patient information, demographics, and other text entered during the call or entered within a message. The communication portal 220 may be implemented as a medical telephone operator (e.g., clinic receptionist, telehealth operator), an internet-based web portal (e.g., hospital website), a message service (e.g., text-based phone short message service (SMS) messaging, smartphone application interface), or another interactive communication service. In an example, the communication portal 220 may include a computer-based virtual operator that plays voice prompts to the patient to prompt for the initial medical information and additional needed medical information, such as using text-to-speech (TTS) or voice-to-text) to interact by voice with the patient.

Instead of directly relaying the information to a clinician 270, the information may be routed to a rules engine 230 (e.g., inference engine) with a medical knowledge base. The rules engine 230 may be implemented as an automated (e.g., computerized) expert system. This expert system may use a rules-based framework that analyzes the message text and incorporates algorithmic techniques to ask additional relevant questions to enable a medical practitioner to provide medical care for the medical event. The expert system may also use the rules-based framework to analyze the message text and incorporates algorithmic techniques to ask additional relevant questions to enable the expert system to determine the urgency or severity of the patient's medical situation.

If the rules engine 230 determines that more information is needed to determine the urgency, then the engine may generate additional questions 240 based on the previously provided input. In an example, additional clarifying clinical questions may be generated based on each previous answer. These additional questions may prompt the medical contact center operator or patient to provide responses 250 to the additional questions. These additional responses may be further analyzed by the rules engine 230 to determine whether more information is needed to identify and describe the medical event or to determine the urgency. Once the rules engine 230 determines that enough information has been gathered for the medical event, the answers 260 may be documented and the information relayed to the clinician 270. This provides the ability for the medical message relay 200 to relay information with accurate symptoms, medical severity, and response urgency.

The knowledge base used in the rules engine 230 may include information to help predict the seriousness (e.g., severity) of the call. The knowledge base may also include varying degrees of information needed for appropriate triage to expedite the communication between the medical clinician and the patient. The knowledge base may be modified and extended through a knowledge base editor, which provides an interface for the operator of the improved system disclosed herein, and also for third-party collaborators. A knowledge base editor may be overseen by human clinical experts that will have the authority to validate and modify the knowledge base, such as to determine when a portion of spoken or written text may be used to determine severity or urgency. The knowledge base editor may be improved over time either manually (e.g., editing by a human clinical expert) or automatically (e.g., by retraining a machine learning model using additional data). These improvements may include adding medical knowledge as new medical discoveries and medical advances are made over time regarding patient callers. The inventive system may also include self-learning capabilities and the ability to add to the knowledge base based on user feedback from a medical provider.

The rules engine may implement algorithmic techniques, and may use decision trees. The decision trees may be arranged to begin with general and broad medical text to increasingly narrow and specific questions down the decision tree. The output of the rules engine may be channeled in analysis module and cross-referenced with the data in the knowledge base, which may be used to send a message to the medical provider and associated clinical recommendations for the medical event description and urgency of the case that is output by the reporting engine. The rules engine may be modified and extended through the rules engine editor, which provides an interface not only for the operator of the expert system disclosed herein, but also for third-party collaborators.

The rules editor may be overseen by human clinical and information technology (IT) experts, who may have the authority to validate and modify the rules engine. The rules engine may also be set to be self-learning by incorporating user feedback entered by providers, such as by providers who may agree or disagree with the description of the medical event, medical symptoms, response urgency, clinical recommendations, or other medical information produced by the inventive system. In an example, the use of self-learning may be implemented as an option of the system that can be turned off depending on provider preference. The rules engine editor users may have the ability to add and or modify the rule sets and AI algorithms in the rules engine over time. The inventive system may also self-learn to reinforce or deemphasize various weights of each of the algorithms, such using new clinical research and user feedback from providers.

Various interpretive results modules may be used for outputting the results of the present subject matter. In an example, a first interpretative results module may be used for healthcare providers, and a second interpretative results module may be used for patients. A provider interface may send all the information and clinical recommendations produced by the analysis module to the provider. The patient interface may send all or a subset of the diagnostic impressions and clinical recommendations produced by the analysis to the patient using secure, HIPAA compliant, real-time electronic means, such as through a bidirectional communications path to the patient using one or more secure means of communication.

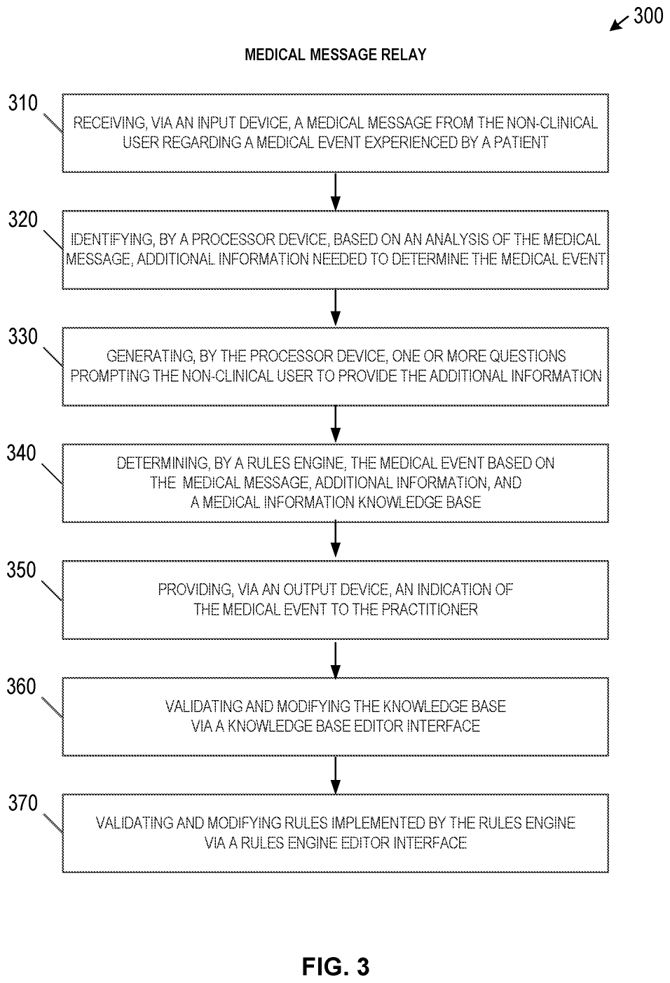

FIG. 3 is a flowchart diagram of a message relay method 300, according to an embodiment. Method 300 includes receiving 310, via an input device, a medical message from the non-clinical user regarding a medical event experienced by a patient. Method 300 further includes identifying 320, by a processor device, based on an analysis of the medical message, additional information needed to determine the medical event or medical event severity. Method 300 further includes generating 330, by the processor device, one or more questions prompting the non-clinical user to provide the additional information. Method 300 further includes determining 340, by a rules engine, the medical event based on the medical message, the additional information, and a knowledge base containing medical information for predicting severity of medical events. Method 300 further includes providing 350, via an output device, an indication of the medical event to the practitioner.

The processor device may use algorithmic techniques to identify the additional information based on analyzing the medical message. The algorithmic techniques may include probabilistic techniques, heuristic techniques, deterministic techniques, and other analytical methods. The rules engine may implement algorithmic techniques using decision trees to determine the medical event. The decision trees may be arranged to analyze the medical message and additional information to determine the medical event.

Method 300 may further include validating and modifying 360 the knowledge base via a knowledge base editor interface. Clinical experts may oversee the knowledge base editor to ensure the knowledge base is kept up to date. Method 300 may further include validating and modifying 370 rules implemented by the rules engine via a rules engine editor interface. Clinical and information technology experts may oversee the rules engine editor.

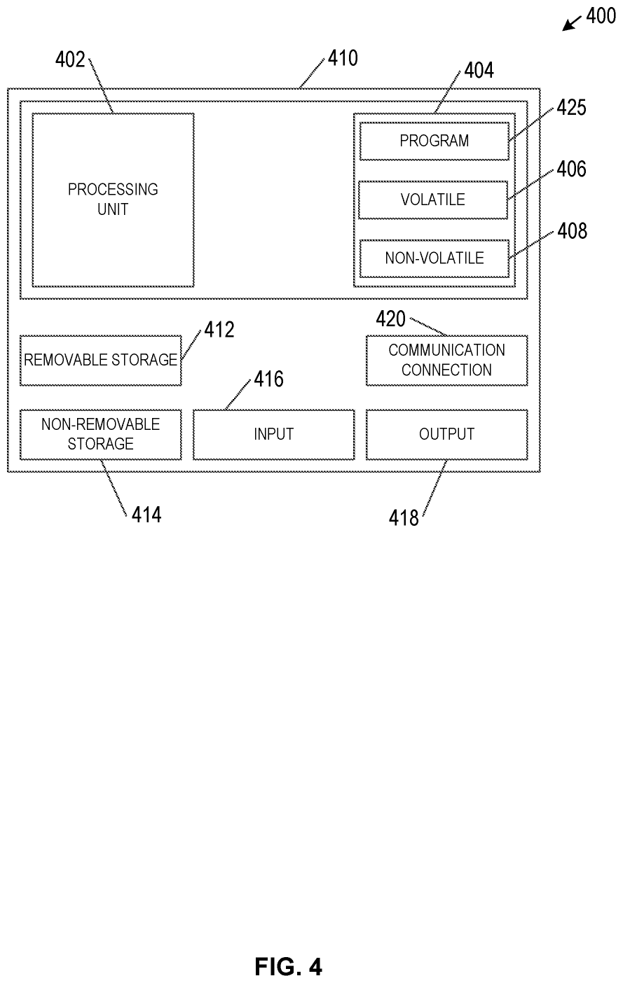

FIG. 4 is a block diagram of a computing device 400, according to an embodiment. The performance of one or more components within computing device 400 may be improved by including one or more of the circuits or circuitry methods described herein. Computing device 400 may include a system for relaying a medical message, according to an embodiment. In one embodiment, multiple such computer systems are used in a distributed network to implement multiple components in a transaction-based environment. An object-oriented, service-oriented, or other architecture may be used to implement such functions and communicate between the multiple systems and components. In some embodiments, the computing device of FIG. 4 is an example of a client device that may invoke methods described herein over a network. In some embodiments, the computing device of FIG. 4 is an example of one or more of the personal computer, smartphone, tablet, or various servers.

One example computing device in the form of a computer 410, may include a processing unit 402, memory 404, removable storage 412, and non-removable storage 414. Although the example computing device is illustrated and described as computer 410, the computing device may be in different forms in different embodiments. For example, the computing device may instead be a smartphone, a tablet, or other computing device including the same or similar elements as illustrated and described with regard to FIG. 4. Further, although the various data storage elements are illustrated as part of the computer 410, the storage may include cloud-based storage accessible via a network, such as the Internet.

Returning to the computer 410, memory 404 may include volatile memory 406 and non-volatile memory 408. Computer 410 may include or have access to a computing environment that includes a variety of computer-readable media, such as volatile memory 406 and non-volatile memory 408, removable storage 412 and non-removable storage 414. Computer storage includes random access memory (RAM), read only memory (ROM), erasable programmable read-only memory (EPROM) & electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions. Computer 410 may include or have access to a computing environment that includes input 416, output 418, and a communication connection 420. The input 416 may include one or more of a touchscreen, touchpad, mouse, keyboard, camera, and other input devices. The input 416 may include a navigation sensor input, such as a GNSS receiver, a SOP receiver, an inertial sensor (e.g., accelerometers, gyroscopes), a local ranging sensor (e.g., LIDAR), an optical sensor (e.g., cameras), or other sensors. The computer may operate in a networked environment using a communication connection 420 to connect to one or more remote computers, such as database servers, web servers, and another computing device. An example remote computer may include a personal computer (PC), server, router, network PC, a peer device or other common network node, or the like. The communication connection 420 may be a network interface device such as one or both of an Ethernet card and a wireless card or circuit that may be connected to a network. The network may include one or more of a Local Area Network (LAN), a Wide Area Network (WAN), the Internet, and other networks.

Computer-readable instructions stored on a computer-readable medium are executable by the processing unit 402 of the computer 410. A hard drive (magnetic disk or solid state), CD-ROM, and RAM are some examples of articles including a non-transitory computer-readable medium. For example, various computer programs 425 or apps, such as one or more applications and modules implementing one or more of the methods illustrated and described herein or an app or application that executes on a mobile device or is accessible via a web browser, may be stored on a non-transitory computer-readable medium.

The apparatuses and methods described above may include or be included in high-speed computers, communication and signal processing circuitry, single-processor module or multi-processor modules, single embedded processors or multiple embedded processors, multi-core processors, message information switches, and application-specific modules including multilayer or multi-chip modules. Such apparatuses may further be included as sub-components within a variety of other apparatuses (e.g., electronic systems), such as televisions, cellular telephones, personal computers (e.g., laptop computers, desktop computers, handheld computers, etc.), tablets (e.g., tablet computers), workstations, radios, video players, audio players (e.g., MP3 (Motion Picture Experts Group, Audio Layer 3) players), vehicles, medical devices (e.g., heart monitors, blood pressure monitors, etc.), set top boxes, and others.

In the detailed description and the claims, the term “on” used with respect to two or more elements (e.g., materials), one “on” the other, means at least some contact between the elements (e.g., between the materials). The term “over” means the elements (e.g., materials) are in close proximity, but possibly with one or more additional intervening elements (e.g., materials) such that contact is possible but not required. Neither “on” nor “over” implies any directionality as used herein unless stated as such.

In the detailed description and the claims, a list of items joined by the term “at least one of” may mean any combination of the listed items. For example, if items A and B are listed, then the phrase “at least one of A and B” means A only; B only; or A and B. In another example, if items A, B, and C are listed, then the phrase “at least one of A, B and C” means A only; B only; C only; A and B (excluding C); A and C (excluding B); B and C (excluding A); or all of A, B, and C. Item A may include a single element or multiple elements. Item B may include a single element or multiple elements. Item C may include a single element or multiple elements.

In the detailed description and the claims, a list of items joined by the term “one of” may mean only one of the list items. For example, if items A and B are listed, then the phrase “one of A and B” means A only (excluding B), or B only (excluding A). In another example, if items A, B, and C are listed, then the phrase “one of A, B and C” means A only; B only; or C only. Item A may include a single element or multiple elements. Item B may include a single element or multiple elements. Item C may include a single element or multiple elements.

### Additional Notes and Examples

Example 1 is a system for increasing accuracy of medical information relayed to a medical practitioner, the system comprising: a storage device, the storage device including a medical knowledge base; an input device configured to receive a medical message from a patient regarding a medical event experienced by the patient; and a processing circuitry configured to: identify, based on an analysis of the medical message, additional information needed by a medical practitioner to provide medical care for the medical event; generate one or more questions prompting the patient to provide the additional information; and implement a rules engine configured to determine the medical event based on the medical message, the additional information, and the medical knowledge base; and provide an indication of the medical event to a medical practitioner.

In Example 2, the subject matter of Example 1 includes, wherein: the processing circuitry is further configured to identify additional urgency information needed to determine an urgency of the medical event and generate one or more questions prompting patient to provide the additional urgency information; the rules engine is further configured to determine the urgency of the medical event based on the medical message, the additional information, and the medical knowledge base, wherein the medical knowledge base includes medical information for predicting severity of medical events; the processing circuitry is further configured to provide an indication of the urgency to a medical practitioner.

In Example 3, the subject matter of Examples 1-2 includes, wherein the processing circuitry uses algorithmic techniques to identify, based on an analysis of the medical message, the additional information.

In Example 4, the subject matter of Examples 1-3 includes, wherein the rules engine implements algorithmic techniques using decision trees.

In Example 5, the subject matter of Examples 1-4 includes, the processing circuitry further configured to: present a knowledge base editor interface; receive a rule modification input at the knowledge base editor interface; and generate an updated medical knowledge base based on the rule modification input.

In Example 6, the subject matter of Examples 1-5 includes, the processing circuitry further configured to: present a rules engine editor interface; receive a rule modification input at the rules engine editor interface; and generate an updated rules engine based on the rule modification input.

In Example 7, the subject matter of Examples 1-6 includes, wherein identifying of the additional information further includes application of probabilistic techniques to analyze the medical message.

In Example 8, the subject matter of Example 7 includes, wherein the probabilistic techniques include statistical predictions.

In Example 9, the subject matter of Examples 1-8 includes, wherein identifying of the additional information further includes application of heuristic techniques to analyze the medical message.

In Example 10, the subject matter of Example 9 includes, wherein the heuristic techniques include rapid approximations.

In Example 11, the subject matter of Examples 1-10 includes, wherein identifying of the additional information further includes application of deterministic techniques to analyze the medical message.

In Example 12, the subject matter of Example 11 includes, wherein the deterministic techniques include following a flowchart.

In Example 13, the subject matter of Examples 1-12 includes, wherein identifying of the additional information further includes application of generative artificial intelligence to generate the one or more questions.

In Example 14, the subject matter of Examples 1-13 includes, wherein identifying of the additional information further includes application of machine learning to generate the one or more questions.

In Example 15, the subject matter of Examples 1-14 includes, wherein identifying of the additional information further includes application of an expert system to generate the one or more questions.

In Example 16, the subject matter of Examples 1-15 includes, wherein identifying of the additional information further includes application of internet-based communications to generate the one or more questions.

Example 17 is a method for increasing accuracy of medical information relayed to a medical practitioner, the method comprising: receiving, via an input device, a medical message from a patient regarding a medical event experienced by the patient; identifying, by a processing circuitry, based on an analysis of the medical message, additional information needed by a medical practitioner to provide medical care for the medical event; generating, by the processing circuitry, one or more questions prompting the patient to provide the additional information; determining, by a rules engine, the medical event based on the medical message, the additional information, and a medical knowledge base containing medical information for predicting severity of medical events; and providing an indication of the medical event to a medical practitioner.

In Example 18, the subject matter of Example 17 includes, identifying, at the processing circuitry, additional urgency information needed to determine an urgency of the medical event and generate one or more questions prompting patient to provide the additional urgency information; and determining, at the rules engine, the urgency of the medical event based on the medical message, the additional information, and the medical knowledge base, wherein the medical knowledge base includes medical information for predicting severity of medical events; providing an indication of the urgency to a medical practitioner.

In Example 19, the subject matter of Examples 17-18 includes, wherein the processing circuitry uses algorithmic techniques to identify the additional information based on analyzing the medical message.

In Example 20, the subject matter of Examples 17-19 includes, wherein the rules engine implements algorithmic techniques using decision trees to determine the medical event.

In Example 21, the subject matter of Examples 17-20 includes, presenting a knowledge base editor interface; receiving a rule modification input at the knowledge base editor interface; and generating an updated medical knowledge base based on the rule modification input.

In Example 22, the subject matter of Examples 17-21 includes, presenting a rules engine editor interface; receiving a rule modification input at the rules engine editor interface; and generating an updated rules engine based on the rule modification input.

In Example 23, the subject matter of Examples 17-22 includes, wherein identifying of the additional information further includes application of probabilistic techniques to analyze the medical message.

In Example 24, the subject matter of Example 23 includes, wherein the probabilistic techniques include statistical predictions.

In Example 25, the subject matter of Examples 17-24 includes, wherein identifying of the additional information further includes application of heuristic techniques to analyze the medical message.

In Example 26, the subject matter of Example 25 includes, wherein the heuristic techniques include rapid approximations.

In Example 27, the subject matter of Examples 17-26 includes, wherein identifying of the additional information further includes application of deterministic techniques to analyze the medical message.

In Example 28, the subject matter of Example 27 includes, wherein the deterministic techniques include following a flowchart.

In Example 29, the subject matter of Examples 17-28 includes, wherein identifying of the additional information further includes application of generative artificial intelligence to generate the one or more questions.

In Example 30, the subject matter of Examples 17-29 includes, wherein identifying of the additional information further includes application of machine learning to generate the one or more questions.

In Example 31, the subject matter of Examples 17-30 includes, wherein identifying of the additional information further includes application of an expert system to generate the one or more questions.

In Example 32, the subject matter of Examples 17-31 includes, wherein identifying of the additional information further includes application of internet-based communications to generate the one or more questions.

Example 33 is a non-transitory machine-readable storage medium, comprising instructions that, responsive to being executed with processor circuitry of a computer-controlled device, cause the processor circuitry to: receive, via an input device, a medical message from a patient regarding a medical event experienced by a patient; identify, by a processing circuitry, based on an analysis of the medical message, additional information needed by a medical practitioner to provide medical care for the medical event; generate, by the processing circuitry, one or more questions prompting the patient to provide the additional information; determine, by a rules engine, the medical event based on the medical message, the additional information, and a medical knowledge base containing medical information for predicting severity of medical events; and provide, via an output device, an indication of the medical event to a medical practitioner.

In Example 34, the subject matter of Example 33 includes, the instructions further causing the processor circuitry to: identify additional urgency information needed to determine an urgency of the medical event and generate one or more questions prompting patient to provide the additional urgency information; and determine the urgency of the medical event based on the medical message, the additional information, and the medical knowledge base, wherein the medical knowledge base includes medical information for predicting severity of medical events; provide an indication of the urgency to a medical practitioner.

In Example 35, the subject matter of Examples 33-34 includes, wherein the processing circuitry uses algorithmic techniques to identify the additional information based on analyzing the medical message.

In Example 36, the subject matter of Examples 33-35 includes, wherein the rules engine implements algorithmic techniques using decision trees to determine the medical event.

In Example 37, the subject matter of Examples 33-36 includes, the instructions further causing the processor circuitry to: present a knowledge base editor interface; receive a rule modification input at the knowledge base editor interface; and generate an updated medical knowledge base based on the rule modification input.

In Example 38, the subject matter of Examples 33-37 includes, the instructions further causing the processor circuitry to: present a rules engine editor interface; receive a rule modification input at the rules engine editor interface; and generate an updated rules engine based on the rule modification input.

In Example 39, the subject matter of Examples 33-38 includes, wherein identifying of the additional information further includes application of probabilistic techniques to analyze the medical message.

In Example 40, the subject matter of Example 39 includes, wherein the probabilistic techniques include statistical predictions.

In Example 41, the subject matter of Examples 33-40 includes, wherein identifying of the additional information further includes application of heuristic techniques to analyze the medical message.

In Example 42, the subject matter of Example 41 includes, wherein the heuristic techniques include rapid approximations.

In Example 43, the subject matter of Examples 33-42 includes, wherein identifying of the additional information further includes application of deterministic techniques to analyze the medical message.

In Example 44, the subject matter of Example 43 includes, wherein the deterministic techniques include following a flowchart.

In Example 45, the subject matter of Examples 33-44 includes, wherein identifying of the additional information further includes application of generative artificial intelligence to generate the one or more questions.

In Example 46, the subject matter of Examples 33-45 includes, wherein identifying of the additional information further includes application of machine learning to generate the one or more questions.

In Example 47, the subject matter of Examples 33-46 includes, wherein identifying of the additional information further includes application of an expert system to generate the one or more questions.

In Example 48, the subject matter of Examples 33-47 includes, wherein identifying of the additional information further includes application of internet-based communications to generate the one or more questions.

Example 49 is at least one machine-readable medium including instructions that, when executed by processing circuitry, cause the processing circuitry to perform operations to implement of any of Examples 1-48.

Example 50 is an apparatus comprising means to implement of any of Examples 1-48.

Example 51 is a system to implement of any of Examples 1-48.

Example 52 is a method to implement of any of Examples 1-48.

The subject matter of any Examples above may be combined in any combination.

The above description and the drawings illustrate some embodiments of the inventive subject matter to enable those skilled in the art to practice the embodiments of the inventive subject matter. Other embodiments may incorporate structural, logical, electrical, process, and other changes. Examples merely typify possible variations. Portions and features of some embodiments may be included in, or substituted for, those of others. Many other embodiments will be apparent to those of skill in the art upon reading and understanding the above description.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A system for increasing accuracy of medical information relayed to a medical practitioner, the system comprising:
a storage device, the storage device including a medical knowledge base;
an input device configured to receive a medical message from a patient regarding a medical event experienced by the patient; and
a processing circuitry configured to:
identify, based on an analysis of the medical message, additional information needed by a medical practitioner to provide medical care for the medical event;
generate one or more questions prompting the patient to provide the additional information; and
implement a rules engine configured to determine the medical event based on the medical message, the additional information, and the medical knowledge base; and
provide an indication of the medical event to a medical practitioner.

2. The system of claim 1, wherein:
the processing circuitry is further configured to identify additional urgency information needed to determine an urgency of the medical event and generate one or more questions prompting patient to provide the additional urgency information;
the rules engine is further configured to determine the urgency of the medical event based on the medical message, the additional information, and the medical knowledge base, wherein the medical knowledge base includes medical information for predicting severity of medical events;
the processing circuitry is further configured to provide an indication of the urgency to a medical practitioner.

3. The system of claim 1, wherein the processing circuitry uses algorithmic techniques to identify, based on an analysis of the medical message, the additional information.

4. The system of claim 1, wherein the rules engine implements algorithmic techniques using decision trees.

5. The system of claim 1, the processing circuitry further configured to:
present a knowledge base editor interface;
receive a rule modification input at the knowledge base editor interface; and
generate an updated medical knowledge base based on the rule modification input.

6. The system of claim 1, the processing circuitry further configured to:
present a rules engine editor interface;
receive a rule modification input at the rules engine editor interface; and
generate an updated rules engine based on the rule modification input.

7. The system of claim 1, wherein identifying of the additional information further includes application of probabilistic techniques to analyze the medical message.

8. The system of claim 1, wherein identifying of the additional information further includes application of heuristic techniques to analyze the medical message.

9. The system of claim 1, wherein identifying of the additional information further includes application of deterministic techniques to analyze the medical message.

10. A method for increasing accuracy of medical information relayed to a medical practitioner, the method comprising:
receiving, via an input device, a medical message from a patient regarding a medical event experienced by the patient;
identifying, by a processing circuitry, based on an analysis of the medical message, additional information needed by a medical practitioner to provide medical care for the medical event;
generating, by the processing circuitry, one or more questions prompting the patient to provide the additional information;
determining, by a rules engine, the medical event based on the medical message, the additional information, and a medical knowledge base containing medical information for predicting severity of medical events; and
providing an indication of the medical event to a medical practitioner.

11. The method of claim 10, further including:
identifying, at the processing circuitry, additional urgency information needed to determine an urgency of the medical event and generate one or more questions prompting patient to provide the additional urgency information; and
determining, at the rules engine, the urgency of the medical event based on the medical message, the additional information, and the medical knowledge base, wherein the medical knowledge base includes medical information for predicting severity of medical events;
providing an indication of the urgency to a medical practitioner.

12. The method of claim 10, wherein the processing circuitry uses algorithmic techniques to identify the additional information based on analyzing the medical message.

13. The method of claim 10, wherein the rules engine implements algorithmic techniques using decision trees to determine the medical event.

14. The method of claim 10, further including:
presenting a knowledge base editor interface;
receiving a rule modification input at the knowledge base editor interface; and
generating an updated medical knowledge base based on the rule modification input.

15. The method of claim 10, wherein identifying of the additional information further includes application of probabilistic techniques to analyze the medical message.

16. A non-transitory machine-readable storage medium, comprising instructions that, responsive to being executed with processor circuitry of a computer-controlled device, cause the processor circuitry to:
receive, via an input device, a medical message from a patient regarding a medical event experienced by a patient;
identify, by a processing circuitry, based on an analysis of the medical message, additional information needed by a medical practitioner to provide medical care for the medical event;
generate, by the processing circuitry, one or more questions prompting the patient to provide the additional information;
determine, by a rules engine, the medical event based on the medical message, the additional information, and a medical knowledge base containing medical information for predicting severity of medical events; and
provide, via an output device, an indication of the medical event to a medical practitioner.

17. The non-transitory machine-readable storage medium of claim 16, the instructions further causing the processor circuitry to:
identify additional urgency information needed to determine an urgency of the medical event and generate one or more questions prompting patient to provide the additional urgency information; and
determine the urgency of the medical event based on the medical message, the additional information, and the medical knowledge base, wherein the medical knowledge base includes medical information for predicting severity of medical events;
provide an indication of the urgency to a medical practitioner.

18. The non-transitory machine-readable storage medium of claim 16, wherein the processing circuitry uses algorithmic techniques to identify the additional information based on analyzing the medical message.

19. The non-transitory machine-readable storage medium of claim 16, wherein the rules engine implements algorithmic techniques using decision trees to determine the medical event.

20. The non-transitory machine-readable storage medium of claim 16, the instructions further causing the processor circuitry to:
present a rules engine editor interface;
receive a rule modification input at the rules engine editor interface; and
generate an updated rules engine based on the rule modification input.
